(19) United States
(12) Patent Application Publication
(10) Pub. No.: US 2010/0160731 A1
(43) Pub. Date: Jun. 24, 2010

(54) ULTRASOUND-VISUALIZABLE ENDOSCOPIC ACCESS SYSTEM

(76) Inventors: Marc Giovannini, Marseille Cedex (FR); Darach McGrath, Tipperary (IE)

Correspondence Address: BRINKS HOFER GILSON & LIONE/CHICAGO/COOK, PO BOX 10395, CHICAGO, IL, 60610, US

(21) Appl. No.: 12/636,439

(22) Filed: Dec. 11, 2009

Related U.S. Application Data
(60) Provisional application No. 61/139,706, filed on Dec. 22, 2008.

(51) Int. Cl. A61B 1/00

(52) U.S. Cl. 600/117

(57) ABSTRACT

An endoscopic access system is provided including an outer cannula and a penetrating stylet. The stylet provides an enhanced echogenic profile configured to provide for effective navigation under ultrasound visualization in a patient body. The cannula may include a rounded distal margin configured to provide for efficient passage though the cannula distal end of tools such as a wire guide.

### CROSS-REFERENCE TO RELATED APPLICATIONS

This application claims priority to U.S. Provisional Application Ser. No. 61/139,706, filed Dec. 22, 2008, which is incorporated herein by reference in its entirety.

### TECHNICAL FIELD

The invention relates generally to medical devices. More particularly, the invention pertains to an access device such as a cannula including means for ultrasound visualization of same.

### BACKGROUND

The development of minimally invasive methods and devices over recent years has revolutionized the practice of medicine. These methods and devices allow clinicians to perform a wide variety of procedures while minimizing trauma to the patient. Along these lines, there is a need for devices and methods which employ minimally invasive technologies in order to access occluded regions in a mammalian body that may not be visible even from a minimally invasive device such as endoscope. This is a particular challenge when a target region needs to be identified with sufficient locational specificity that visualization is preferable, but video visualization (e.g., via a camera or other video element of an endoscope) may be impractical, and it is generally desirable to minimize the use of fluoroscopic visualization particularly for an extended time period as may be required to navigate a cannula to a target site.

Some prior art catheter, cannula, or similar access devices are difficult to navigate to a precise location within the body. This often occurs due to the lack of a guidance system configured to indicate where the device is located without introducing the risks associated with over-use of fluoroscopy. It would be beneficial to provide minimally access devices and methods for their use which limit one or more of these or other problems.

### BRIEF SUMMARY

An endoscopic access system is provided including an outer cannula and a penetrating stylet. The stylet provides an enhanced echogenic profile configured to provide for effective navigation under ultrasound visualization in a patient body. The cannula may include a rounded distal margin configured to provide for efficient passage of tools such as a wire guide though the cannula distal end.

In one embodiment, an endoscopic access system includes a flexible outer cannula with a first cannula lumen extending from a distal end toward a proximal end of the cannula; and a stylet disposed removably through the first cannula lumen, the stylet including a piercing distal tip, a flexible body length extending proximally from the piercing distal tip, and an echogenic stylet portion disposed immediately adjacent the piercing distal tip, the echogenic stylet portion configured to provide reflection of ultrasonic waves sufficient for ultrasonic imaging of the echogenic stylet portion at a resolution providing for effective navigation the cannula in a body.

### BRIEF DESCRIPTION OF THE DRAWINGS

The invention may be better understood with reference to the following drawings and description. The components in the figures are not necessarily drawn to scale, emphasis instead being placed upon illustrating the principles of the invention.

FIG. 1 shows an embodiment of an ultrasound-visualizable endoscopic access system;

FIGS. 2A-2C show a method of using an ultrasound-visualizable endoscopic access system with an echogenic stylet;

FIG. 3 shows another embodiment of an ultrasound-visualizable endoscopic access system;

FIGS. 4A-4B show a method of using an ultrasound-visualizable endoscopic access system with an echogenic cannula;

FIG. 5 shows another embodiment of an ultrasound-visualizable endoscopic access system;

FIG. 6 shows a multi-lumen embodiment of an ultrasound-visualizable endoscopic access system;

FIG. 7 shows another multi-lumen embodiment of an ultrasound-visualizable endoscopic access system;

FIG. 8 shows, in section view, a current cannula with a tool extending therethrough, having sustained damage from the cannula;

FIG. 9 shows a bevel-tipped cannula embodiment including a rounded distal margin;

FIG. 9A shows a longitudinal section view taken along line A-A of FIG. 9;

FIG. 10 shows a cannula embodiment including a rounded distal margin; and

FIG. 10A shows a longitudinal section view taken along line A-A of FIG. 10.

### DETAILED DESCRIPTION

As used herein, including in the claims, the term “echogenic” is defined as having enhanced echogenicity. Specifically, it is used to refer to materials or portions of materials that are constructed or are treated to have greater reflectivity of ultrasonic waves than standard materials used for a cannula/catheter and/or stylet. It is known in the art that most materials used for a cannula or stylet will reflect some ultrasonic waves, but the term “echogenicity,” as used herein includes treating the surface with dimples, divots, or the like, (and/or, when specifically referenced, using a material known to provide an enhanced echogenic profile) configured to provide clear ultrasound visualization at a resolution providing for accurate location and navigation of a device in a body (e.g., of a patient).

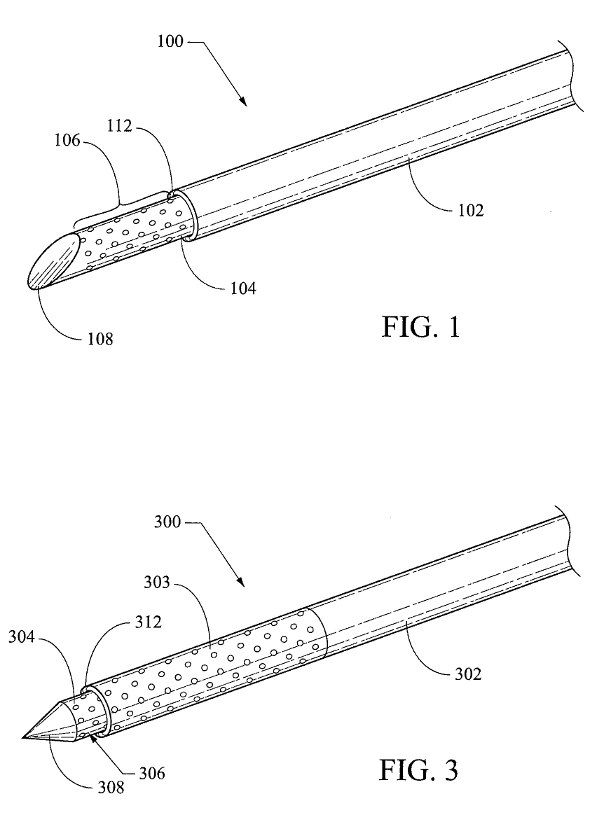

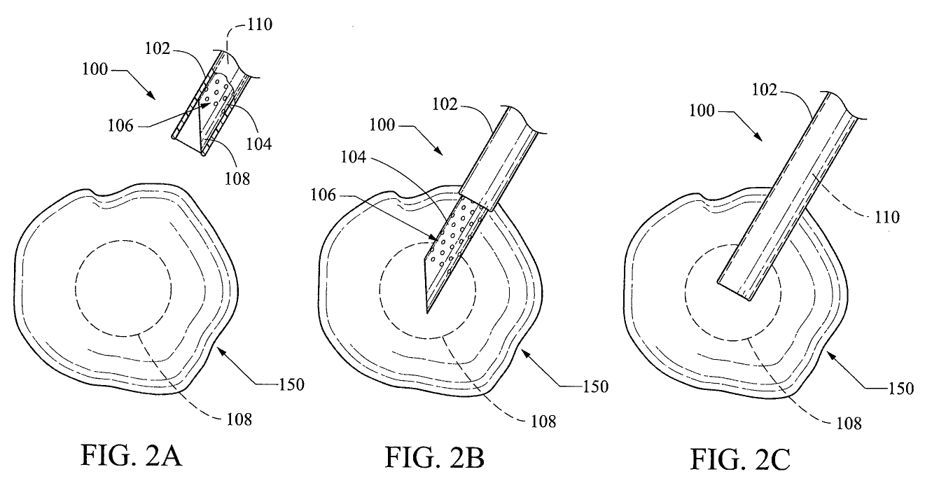

A first embodiment of an ultrasound-visualizable endoscopic access system 100 is described with reference to FIGS. 1-2C. The system includes an elongate flexible outer cannula 102, which includes a cannula lumen 110 extending from a rounded distal end 112 toward a proximal end (not shown) of the cannula 102. (As used herein, the term “cannula” includes a flexible elongate tubular medical device where specified as flexible, such as a catheter, and a more rigid device such as a generally rigid needle.) A removable stylet 104 is shown as having passed through and extending distally from the cannula lumen 110. The stylet 104 includes a piercing tip 108 configured for penetrating through an occlusion. The occlusion may be, by way of illustrative example, the exterior of a pancreatic pseudocyst, the wall of the stomach, an intestinal wall, or another artificial or natural structure between an endoscopically-accessible site and a target site, including creation of an orifice for a natural orifice translumenal endoscopic (NOTES) surgical procedure. The stylet 104 may be constructed of an alloy, such as a nickel-titanium alloy including a shape-memory alloy, stainless steel, or it may be constructed of and/or coated with a polymer including, for example, an echogenic polymer such as is described in PCT Pat. App. Publ. WO02/078611 to Wheatley, et al.

The flexibility of at least a portion of the cannula 102 preferably provides pushability and trackability sufficient to allow navigation through a body lumen or other passage without significant risk of crimping or otherwise occluding the lumen 110. In one embodiment, the cannula may be constructed of stainless steel hypotube or a nickel-titanium alloy. As another example, one embodiment may include a polyether block amide (PEBA), PEBAX, poly-ether-ether-ketone (PEEK), ePTFE, PTFE, or PET cannula, and it will be appreciated that other polymeric materials including polymers with braided construction and/or with metallic components may also be used within the scope of this invention. This pushability and trackability will be enhanced by the stylet 104.

In FIGS. 1-2C, the stylet is illustrated as being constructed from an alloy known in the art to provide desirable traits for pushability and trackability, and including a beveled distal tip 108, which may be beveled in a lancet configuration or any number of other beveled configurations which will include any design configured for effectively piercing tissue. A distal, dimpled echogenic region 106 of the stylet 104 is configured to reflect ultrasonic waves for generation of a visualizable image using a medical ultrasound device (e.g., external ultrasound device, endoscopic ultrasound device). In another embodiment, including one where the stylet may be constructed of, or coated with an echogenic polymer, the region 106 may have a different surface configuration other than being dimpled, but most preferably presents an ultrasound-visualizable profile that provides for location (determining the position of) and navigation of the overlying cannula 102. The cannula 102 may also be constructed from a polymer, alloy, or other material such as those known and used for making endoscopically-deployable catheters and cannulas. For embodiments configured to be used in a larger endoscope working channel endoscope (e.g., inner diameter of about 3.7 mm), a polymer tube may be preferable for providing a desirable profile with regard to stiffness, trackability, and pushability, while a polymer or alloy tube (e.g., stainless steel hypotube) may provide desirable traits for use in a smaller working channel (e.g., ID of about 1.5-2.8 mm or less).

FIG. 3 shows another embodiment of an ultrasound-visualizable endoscopic access system 300. The system includes an outer cannula 302, and a stylet 304 extending longitudinally through a lumen 312 of the cannula 302. The stylet 304 includes an echogenic surface 306, what is shown as being dimpled to reflect ultrasound waves sufficient to provide an image from, for example, an endoscopic ultrasound. A distal region of the cannula 302 is also provided with an echogenic region 303. The echogenic region 303 is illustrated as a thin metallic band continuous with the cannula body 302, and including dimples or divots that make it echo-reflective. However, it will be appreciated that an echogenic polymer may be used, or some other echogenic construction providing for ultrasound visualization of the cannula 302. The stylet 304 includes a generally conical distal piercing tip 308.

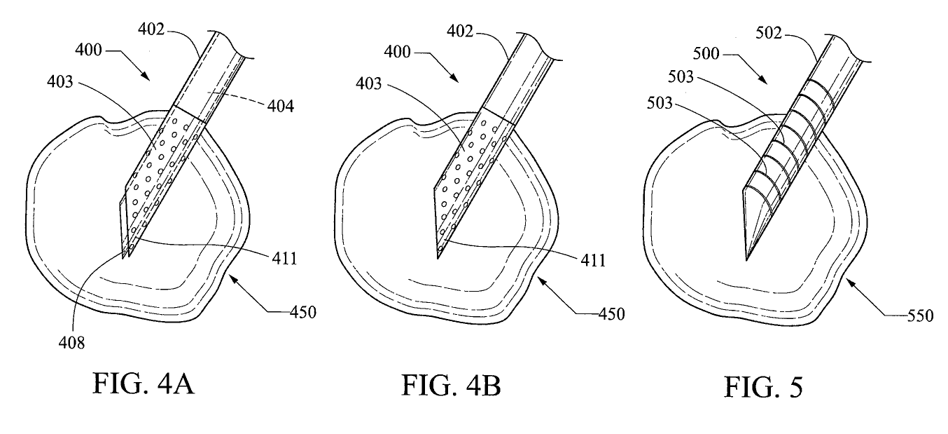

FIGS. 4A and 4B illustrate a method using an ultrasound-visualizable endoscopic access system 400 including a cannula 402 having a distal echogenic region 403. The system includes a stylet 404 extending longitudinally through a lumen of the cannula 402. The stylet 404 includes a generally beveled distal piercing tip 408. The distal region of the cannula 402, with its echogenic region 403, together include a beveled tip 411 which may correspond in angle to the beveled stylet tip 408. The matching beveled tips 408, 411 provide for penetrating access into a target mass 450, which can be carefully guided under ultrasound to a desired location, as shown in FIG. 4A. In a preferred usage, the beveled tips will be aligned, or very nearly aligned (with the stylet tip 408 leading, if they are not aligned) during a piercing operation of penetrating through an occlusion (including entry into a target mass). Then, as shown in FIG. 4B, the stylet may then be withdrawn, after which a therapeutic or diagnostic device or agent (such as, for example, a cytological sampling device, biopsy device, therapeutic solution, or other item or material) may be directed through the cannula 402 to the target site in the mass 450. Expressed another way, the stylet, alone or in combination with the cannula, may function as a needle with an echogenic region that preferably is at or very near the distal end and is thereby configured to provide an ability for using ultrasound for effective visualization during navigation.

FIG. 5 shows another embodiment of an ultrasound-visualizable endoscopic access system 500, disposed in a target mass 550. The system includes a cannula 502 that has a distal echogenic portion. The distal echogenic portion is provided by one or more fiducials 503. The term “fiducials” is here defined to include fiducials and fiducial-like structures of the type known in the art to provide an echogenic profile including, for example, cylindrical slugs of gold, platinum, rhenium, alloys of these, or other materials. Such fiducials are commonly placed in a body to use as a navigational marker for other procedures. However, in this embodiment, the one or more fiducials 503 are embedded in the cannula wall 502. It should be appreciated that the fiducials could, in other embodiments, also or alternatively be disposed on an exterior or interior surface of the cannula wall 502.

A method of use for the system 500 may be understood with reference to FIGS. 4A and 4B and corresponding text, which describes using ultrasound to guide the cannula to a target region. Like the system 400, this embodiment may be used with an echogenic or a non-echogenic stylet, and it is anticipated that the stylet tip may be kept retracted into the cannula lumen during transit through an endoscope working channel to protect both the tip and the channel surface. For an embodiment using a non-echogenic stylet, proximal indicia (e.g., visual and/or tactile indicia at the proximal end of the system being manipulated by a user) may be provided to aid orientation of the relative positions of the stylet and cannula.

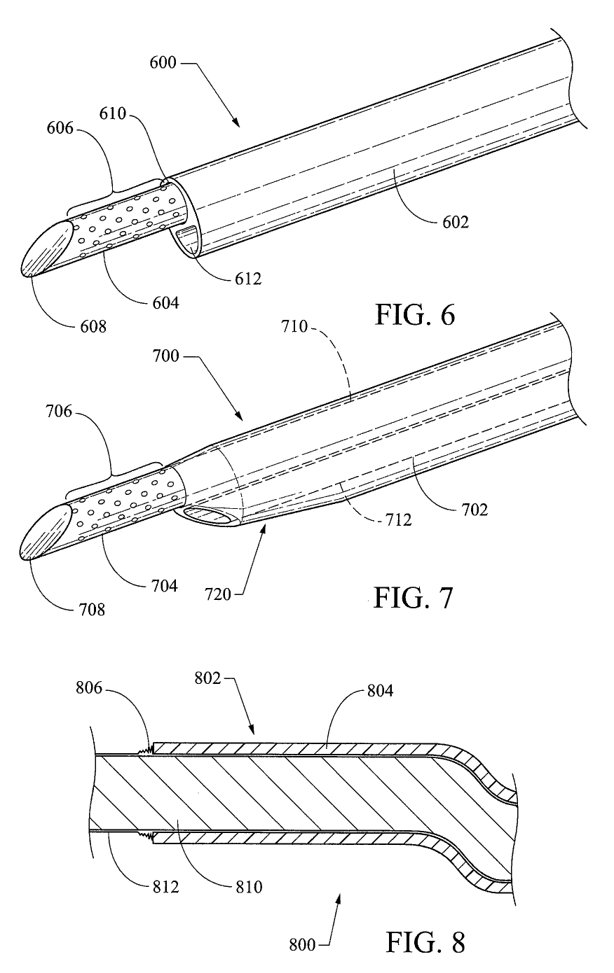

FIG. 6 shows a multi-lumen embodiment of an ultrasound-visualizable endoscopic access system 600. The system includes a flexible outer cannula 602. It also includes a first cannula lumen 610 and a second cannula lumen 612 extending from its distal end toward a proximal end (not shown) of the cannula 602. A stylet 604 is shown as having passed through and extending distally from the first cannula lumen 610. The stylet 604 includes a piercing tip 608 configured for penetrating through an occlusion. A second stylet (not shown) may be provided in the second lumen 612 to, for example, provide enhanced structural strength, prevent entry into the second lumen of extraneous material, and/or some other desired function.

The stylet is illustrated as being constructed from an alloy providing desirable traits for pushability and trackability, and including a beveled distal tip 608, but the tip geometry and material construction may be configured in different manners to suit particular desired applications. A distal, dimpled echogenic region 606 of the stylet 604 is configured to reflect ultrasonic waves for generation of a visualizable image using a medical ultrasound device in the same manner as the embodiments described above with reference to FIG. 1. The second lumen 612 may be used to provide access for a diagnostic or therapeutic device or material without having to remove the stylet 604 from its lumen, and/or both the first and second lumens may be used for the same or different purposes upon removal of the stylet 604. The second lumen 612 may also be used for a wire guide (not shown). In an application of such an embodiment, a wire guide may first be directed toward an occluded target site, preferably getting as near to the target site as permitted by the occlusion and as permitted by a desirable means of visualization of the wire guide, then utilizing the piercing distal tip in an ultrasound-guided manner similar to that described above with reference to FIGS. 2A-2C.

FIG. 7 shows a multi-lumen embodiment of an ultrasound-visualizable endoscopic access system 700. The system includes a flexible outer cannula 702. The cannula 702 has a tapered distal end region 720. It also includes a first cannula lumen 710 and a second cannula lumen 712 extending from its distal end toward a proximal end (not shown) of the cannula 702. A stylet 704 is shown as having been passed through and extending distally from the first cannula lumen 710. The stylet 704 includes a piercing tip 708 configured for penetrating through an occlusion. The tapered distal region 720 of the cannula 702 preferably is configured to follow the piercing tip 708 during an operation of the system 700 where the piercing tip is used to penetrate through an occlusion between an introductory location and a target site. Specifically, the tapered distal region 720 preferably will pass readily through and potentially widen an aperture through an occlusion where the aperture was created and/or initially penetrated by the piercing tip 708, such that the tapered distal region 720 may essentially act as a continuation of the piercing tip. A second stylet (not shown) may be provided in the second lumen 712 to, for example, provide enhanced structural strength, prevent entry into the second lumen of extraneous material, and/or some other desired function.

The stylet is illustrated as being constructed from an alloy providing desirable traits for pushability and trackability, and including a beveled distal tip 708, but the tip geometry and material construction may be configured in different manners to suit particular desired applications. A distal, dimpled echogenic region 706 of the stylet 704, which includes the tip 708, is configured to reflect ultrasonic waves for generation of a visualizable image using a medical ultrasound device in the same manner as the embodiments described above with reference to FIG. 1. The second lumen 712 may be used to provide access for a diagnostic or therapeutic device or material without having to remove the stylet 704 from its lumen, and/or both the first and second lumens may be used for the same or different purposes upon removal of the stylet 704. In other embodiments, the systems 600, 700 of FIGS. 6 and 7 may be configured with an echogenic distal cannula portion, and/or may include more than two lumens.

FIG. 8 shows a longitudinal section view of a known needle access system 800. As shown in FIG. 8, in some current needle-access systems 800 having a stiff or rigid distal portion 802 of a cannula 804, there is an increased risk of damage to a coating 812 of a wire guide 810 or other tool introduced through the lumen of the cannula 804. Specifically, these systems are often configured to have the distal tip 802 of the cannula 804 function as a penetrating needle. As such, the transition from the outer to the inner circumference that forms the distal end/distal margin of the cannula 804 includes one or more sharp or cornered edges 806.

As depicted in FIG. 8, when the outer surface (such as, for example, a lubricious polymer coating) of a wire guide 810 or other tool is moved through the distal cannula opening, that surface can be damaged. In the case of a coating, the coating may be scarred, scored, stripped from the device 810, or otherwise damaged. This damage may not only impair the efficacy of the device 810, but it increases the risk that, as a result of the damage, one or more pieces of the device 810 may become separated from the device 810, which—it will be appreciated—may cause other problems for a patient and/or procedure. Similarly, for a device of the present invention including a treated stylet-surface such as a dimpled or otherwise textured surface configured to enhance echogenicity (including a surface treated with an applied material such as an echogenic polymer), a cannula edge 806 may damage the echogenic surface in a manner adversely affecting its desired echogenic properties.

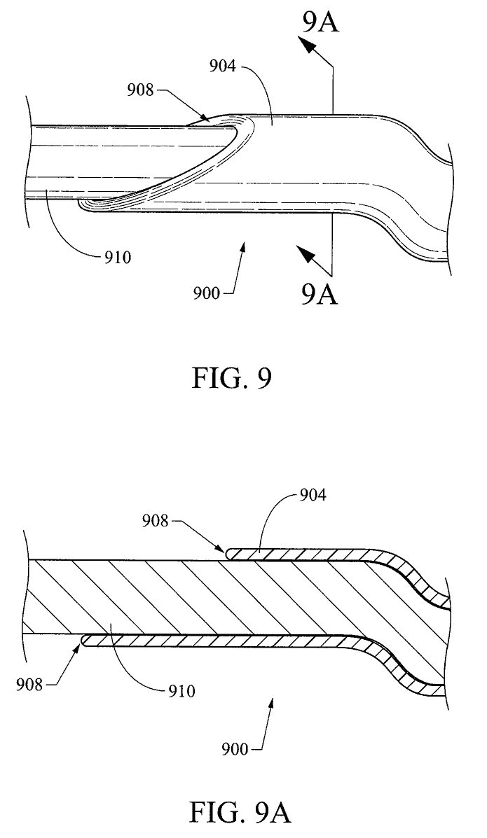

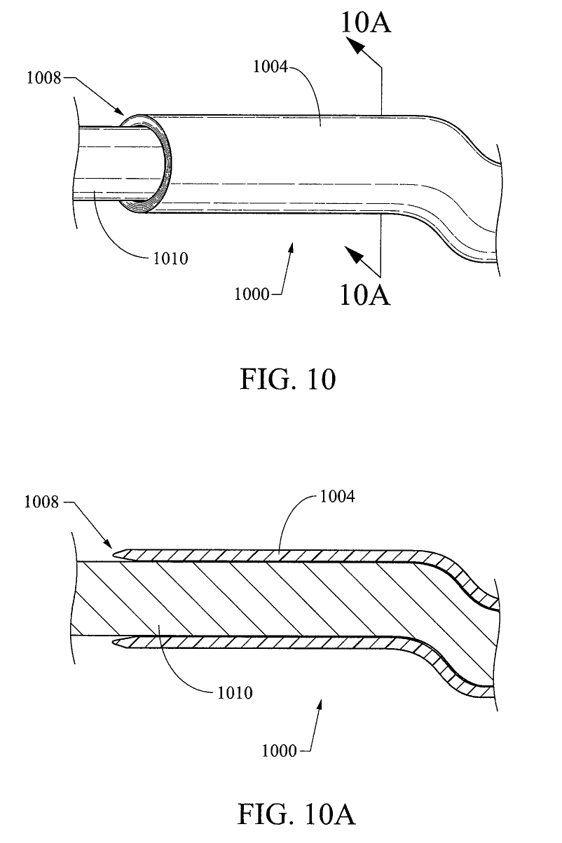

FIG. 9A illustrates, in longitudinal section, a distal portion of a cannula 904 of a cannula access system 900 (taken along line A-A of FIG. 9), with a tool 910 (e.g., stylet, wire guide, or other tool) disposed through its central lumen. FIG. 10A illustrates, in longitudinal section, a distal portion of a cannula 1004 of a needle access system 1000 (taken along line A-A of FIG. 10), with a tool 1010 disposed through its central lumen. The distal end rims 908, 1008 of the cannulas 904, 1004 each have a rounded profile (as viewed in longitudinal section) rather than the cornered or otherwise angled edges present in the previously known devices such as that illustrated and described with reference to FIG. 8. The present inventors surprisingly discovered that providing this rounded profile significantly reduces the likelihood of damage to the outer surface of a tool (specifically the coating of a wire guide, but also applicable, for example, to diagnostic tools, therapeutic tools, etc.) being passed through the cannula. FIGS. 9 and 9A shows a cannula having a beveled (asymmetric) distal end 908, while FIGS. 10 and 10A shows a cannula 1004 having a symmetric distal end 1008. In both embodiments, the distal end forming a transition between the inner circumference and the outer circumference of the cannula is rounded. It will be appreciated that rounding the end margin(s) of a cannula such as a polymeric needle or alloy needle or other cannula type may be done using a laser, a mechanical abrasion/polishing method, a chemical treatment, or other technique known in the art to finely shape materials of the type selected for the cannula.

In the embodiment shown in FIGS. 9-9A, curvature of the cross-section of the cannula wall at its distal end 908 from the inner to the outer circumference is depicted as a regular semi-circle. In the embodiment shown in FIGS. 10-10A, a cross-section of the cannula wall at its distal end 1008 forms a parabola that is asymmetrical (e.g., with a steeper angle nearer the inner circumference). It should be appreciated that other rounded profiles such as, for example, symmetrical parabolas, semi-ellipses, other asymmetrical parabolas may be practiced within the scope of the present invention. The same is true for a cannula where only the transition from the inner circumference to a distal face is rounded, and for a cannula that includes a distal face that is perpendicular or nearly perpendicular to the longitudinal cannula axis with only one edge thereof being rounded. In one exemplary method of use of a system of the present invention including a cannula having a rounded distal end of its defining wall, a sharpened stylet that includes a distal echogenic region will be used to cannulate a path for passage of the rounded-tip cannula, wherein a traumatic (e.g., piercing, cutting) tip of the stylet is directed along a desired path distally from/in advance of the cannula.

Those of skill in the art will appreciate that embodiments not expressly illustrated herein may be practiced within the scope of the present invention, including that features described herein for different embodiments may be combined with each other and/or with currently-known or future-developed technologies while remaining within the scope of the claims presented here. It is therefore intended that the foregoing detailed description be regarded as illustrative rather than limiting. And, it should be understood that the following claims, including all equivalents, are intended to define the spirit and scope of this invention.

1. An ultrasound-visualizable endoscopic access system, comprising:
a flexible outer cannula including a first cannula lumen extending from a distal end toward a proximal end of the cannula; and
a stylet disposed removably through the first cannula lumen, the stylet including
a piercing distal tip;
a flexible body length extending proximally from the piercing distal tip; and
an echogenic stylet portion disposed immediately adjacent the piercing distal tip, the echogenic stylet portion configured to provide reflection of ultrasonic waves sufficient for ultrasonic imaging of the echogenic stylet portion at a resolution providing for effective navigation the cannula in a body.

2. The system of claim 1, further comprising an echogenic cannula portion adjacent the distal end of the outer cannula that is configured to provide reflection of ultrasonic waves sufficient for ultrasonic imaging of the echogenic cannula portion.

3. The system of claim 2, where an ultrasound-reflective material is disposed on the outer surface of the echogenic cannula portion.

4. The system of claim 1, where at least one of the cannula and the stylet comprises an ultrasound-reflective material selected from the group consisting of: a dimpled alloy; an echogenic fiducial; an echogenic polymer; and any combination thereof.

5. The system of claim 1, where the echogenic stylet portion comprises a dimpled surface.

6. The system of claim 1, where the outer cannula comprises an echogenic material.

7. The system of claim 6, where the echogenic material is selected from an echogenic polymer and an alloy metal, the metal having been treated to comprise at least one echogenic surface.

8. The system of claim 1, where the outer cannula comprises a rigid distal portion.

9. The system of claim 1, where the outer cannula further comprises a second cannula lumen disposed generally parallel to the first cannula lumen.

10. The system of claim 1, where a distal end rim of the outer cannula comprises a rounded profile in longitudinal section.

11. The system of claim 1, where the stylet is disposed at least partially through the first cannula lumen.

12. A method for providing endoscopic, ultrasound-guided access to a target site, the method comprising the steps of:
providing the system of claim 1;
directing the echogenic stylet portion and the distal cannula end adjacent a target site obscured by an occlusion, the step of directing comprising determining the location of a cannula portion by ultrasound visualization of the echogenic stylet portion within said cannula portion;
directing the stylet piercing tip to a location nearer the target site; and
directing the distal cannula end along the stylet to a location nearer the target site.

13. The method of claim 12, where access to the target site is at least partially blocked by an occlusion, and the step of directing the stylet piercing tip to a location nearer the target site further comprises directing the stylet piercing tip though the occlusion.

14. The method of claim 13, further comprising directing the cannula distal end nearer the target site.

15. The method of claim 12, where the outer cannula further comprises a second cannula lumen disposed generally parallel to the first cannula lumen and a wire guide aperture providing a path of mechanical communication with a distal portion of the second cannula lumen, the method further comprising the steps of:
directing the distal end of a wire guide to a desired location; and
directing the distal end of the cannula over a proximal end of the wire guide such that the wire guide proximal end passes through the distal portion of the second cannula lumen and out through the wire guide aperture, where the stylet is disposed in a location selected from
in the second cannula lumen, the distal stylet end disposed proximal of the wire guide aperture, and
in the first cannula lumen.

16. The method of claim 12, where the stylet and the cannula are moved simultaneously during at least a portion of the directing steps, with the stylet tip immediately adjacent the distal end of the outer cannula and further comprising a step of withdrawing the stylet from the cannula.

17. The method of claim 12, where the cannula further comprises an echogenic portion adjacent the distal end of the outer cannula, and the step of directing the distal cannula end comprises ultrasound visualization of the cannula echogenic portion.

18. An endoscopic access cannula system comprising:
an outer cannula having a proximal end, a distal end, and a cannula lumen extending proximally from the distal end, where the distal end includes a rounded margin between its outer surface and a surface of the cannula lumen;
a stylet configured for passage through at least a portion of the cannula lumen, where the stylet comprises:
a pointed distal end configured for penetrating tissue to form a path for passage of the cannula; and
a dimpled echogenic surface region immediately adjacent the pointed distal end.

19. The system of claim 18, wherein a distal portion including the distal end of the cannula is rigid.

20. The system of claim 18, further comprising a wire guide disposed through the cannula lumen.
